(19) United States
(12) Patent Application Publication
(10) Pub. No.: US 2022/0047656 A1
(43) Pub. Date: Feb. 17, 2022

(54) SACCHAROMYCES CEREVISIAE YEAST STRAIN FOR THE TREATMENT AND/OR PREVENTION OF OROPHARYNGEAL CANDIDIASIS

(71) Applicant: LESAFFRE ET COMPAGNIE, PARIS (FR)

(72) Inventors: Nathalie BALLET, MARC EN BAROEUL (FR); Amélie DECHERF, BONDUES (FR)

(73) Assignee: LESAFFRE ET COMPAGNIE, PARIS (FR)

(21) Appl. No.: 17/413,716

(22) PCT Filed: Dec. 17, 2019
(86) PCT No.: PCT/EP2019/085473

§ 371 Date: Jun. 14, 2021

(30) Foreign Application Priority Data
Dec. 17, 2018 (FR) 18 73109

(51) Int. Cl. A61K 36/064; C12N 1/18; A61K 45/06; A61K 9/08

(52) U.S. Cl. CPC A61K 36/064; C12N 1/185; C12R 2001/865; A61K 9/08; A61K 45/06

(57) ABSTRACT

The yeast strain Saccharomyces cerevisiae number CNCM I-3856 for the treatment and/or prevention of oropharyngeal infections by Candida. This strain has shown a strong ability to reduce living fungi in the oral cavity and to prevent the propagation of the infection by Candida to the esophagus, the stomach and the small intestine.

### PARENT APPLICATION

The present patent application claims priority of French patent application number FR 18 73107 filed on 17 Dec. 2018. The contents of the French patent application are incorporated by reference in their entirety.

### FIELD OF THE INVENTION

The present invention relates to the field of oropharyngeal infections by Candida. The invention relates more particularly to a specific strain of Saccharomyces cerevisiae useful in the treatment and/or prevention of oral and oropharyngeal candidiasis.

### Context of the Invention

Oropharyngeal candidiasis is a very common mucosal infection, caused in most cases by the fungus Candida albicans, although other species, such as C. glabrata, C. pseudotropicalis, C. krusei, C. parakrusei, and C. guillermondii, may also be the cause of this mycosis. Candida albicans colonizes, in a minor way, the oral flora of healthy subjects without causing infection. However, when this fungus proliferates it causes candidiasis, which is manifested as irritation of the oral mucosae, associated with redness and even ulceration. Sometimes, whitish, more or less pasty spots may be present on the tongue, the palate and sometimes the oropharynx. Oral or oropharyngeal candidiasis may disturb speech, eating, and the quality of life.

Prevalence of oropharyngeal candidiasis is observed in children under 18 months due to immaturity of the immune system and in individuals with reduced flow of saliva leading to dry mouth, such as the elderly, and infants between birth and the 2nd month, before the appearance of salivation. There are also many factors promoting the occurrence of oropharyngeal candidiasis, such as local factors (such as wearing orthodontic or prosthetic devices for a long time); systemic factors such as an immunocompromised state (for example associated with Sjögren syndrome, HIV infection, uncontrolled diabetes, certain endocrine disorders, malnutrition or malabsorption such as for example vitamin B deficiency); and side effects linked to medication (such as treatment with broad spectrum antibiotics, systemic or local use of corticosteroids, administration of neuroleptics, immunosuppressant treatment, chemotherapy or radiotherapy, treatment with a proton pump inhibitor (PPI) or with an H2 antihistamine)

The treatments available for patients with oropharyngeal candidiasis are mainly limited to the use of antifungals. Local treatment (at the level of the mouth), in the form of lozenges, oral suspensions or oral gels, is preferred for most patients. However, in certain cases, in particular for immunodepressed persons or in the case of failure of local treatment, recurrence, or an old infection, systemic treatment, administered by the oral route or by injection, may be necessary.

The antifungals mainly used, to date, for treating oropharyngeal candidiasis are polyene macrolides (nystatin, amphotericin B) and/or azoles (miconazole, fluconazole). Although these drugs are generally well tolerated, they are associated with important drawbacks. Thus, the polyenes, such as amphotericin B, which are effective against most Candida isolates, tend to have a high level of renal toxicity, and combination of them with other drugs such as hypokalemic drugs or nephrotoxic drugs must be avoided. The azole antifungals, in their turn, have common adverse effects such as hepatotoxicity (cytolysis, cholestasis) and digestive disorders (nausea, vomiting, diarrhea). Antifungals of the triazole type (such as fluconazole) are contraindicated in pregnancy, as they are teratogenic. Moreover, since the azoles are all, to varying degrees, enzyme inhibitors of the isoenzymes of CYP450, they cause many drug interactions. Moreover, they themselves are subject to phenomena of metabolization and are therefore also the target of drug interactions.

Finally, a major problem connected with the use of these antifungals is the increasing emergence of resistant strains of Candida albicans (Kelly et al., Lancet, 1996, 348: 1523-1524). Moreover, as the available antifungals are directed either against the “ergosterol” part of the fungal membrane (in the case of the polyenes) or against the enzymes involved in biosynthesis of this ergosterol (in the case of the azoles), there is also a risk of cross resistance (Kelly et al., FEBS Lett., 1997, 400: 80-82; White et al., Clin. Microbiol. Rev., 1998, 11: 382-402).

There is therefore still a need for new strategies for the treatment and/or prevention of oral or oropharyngeal candidiasis.

### SUMMARY OF THE INVENTION

The present inventors found, surprisingly, that the Saccharomyces cerevisiae yeast strain deposited on 17 Oct. 2007 with the CNCM (National Collection of Cultures of Microorganisms, 25 rue du Docteur Roux, 75724 Paris Cedex 15, France) under number I-3856 was able to produce an effective reduction in the burden of Candida albicans in the oral cavity of a murine animal model of oropharyngeal candidiasis and thus prevented spread of the infection to the esophagus, stomach and small intestine. Inhibition of the fungal growth of Candida albicans in the oral cavity was observed when the Saccharomyces cerevisiae yeast I-3856 used in the treatment of the mice is in the form of live yeast or in the form of inactivated yeast. Inhibition of the growth of Candida albicans in the oral cavity was also observed when cell walls of the Saccharomyces cerevisiae yeast I-3856 were used in the treatment of the mice. The beneficial effect of Saccharomyces cerevisiae I-3856 in the live form combines a mechanical effect through strong aggregation of Candida albicans and a biological effect through modulation of the virulence factors of Candida albicans, such as inhibition of adhesins or inhibition of the hyphal transition, which prevent adhesion of the fungal cells to the oral epithelial cells, whereas the effect of Saccharomyces cerevisiae I-3856 in the form of inactivated yeast and of cell walls is mainly only due to a mechanical effect through strong aggregation of Candida albicans, which prevents adhesion of the fungal cells to the oral epithelial cells. All these effects induce an acceleration of the elimination of Candida albicans, which leads to a weak inflammatory response, or even absence of an inflammatory response.

The present invention therefore relates to the Saccharomyces cerevisiae yeast strain deposited with the CNCM on 17 Oct. 2007 under number I-3856, for use in the prevention and/or treatment of oropharyngeal candidiasis.

In certain embodiments, the Saccharomyces cerevisiae yeast strain number I-3856 used in the method of preventing and/or treating oropharyngeal candidiasis is in the live form or the inactive form.

In certain embodiments, the Saccharomyces cerevisiae yeast strain number I-3856 used in the method of preventing and/or treating oropharyngeal candidiasis is in the form of dry yeast, in particular active dry yeast.

In certain embodiments, the Saccharomyces cerevisiae yeast strain number I-3856 used in the method of preventing and/or treating oropharyngeal candidiasis is in the fractionated form. The fractionated form may be selected from the cell walls of said yeast, the wall β-glucans of said yeast, the wall mannoproteins of said yeast, the extracts of said yeast, and any combination thereof.

The present invention also relates to a pharmaceutical composition for use in the prevention and/or treatment of oropharyngeal candidiasis, the pharmaceutical composition comprising an effective amount of the Saccharomyces cerevisiae yeast strain number I-3856, and at least one physiologically acceptable excipient. The Saccharomyces cerevisiae yeast strain number I-3856, present in the pharmaceutical composition, may be in any form defined above.

In certain embodiments, the pharmaceutical composition is intended for topical administration or for administration by the oral route.

In certain embodiments, the pharmaceutical composition further comprises at least one additional active pharmaceutical ingredient having a soothing, anti-irritant, analgesic, anti-inflammatory, wound-healing, antibiotic, antipyretic, or antifungal activity.

In certain embodiments, the pharmaceutical composition is in the form of a toothpaste, a mouthwash, an oral spray, an oral cream or gel, an orodispersible strip, pastilles, a powder which may be dusted directly in the oral cavity, a vial with a push-button stopper, an orodispersible stick, or a stick to be diluted in water.

In certain embodiments, the oropharyngeal candidiasis that is intended to be prevented or treated with the Saccharomyces cerevisiae yeast strain number I-3856, in any form defined above, or else with the pharmaceutical composition defined above, is a side effect of a medical treatment, in particular a treatment with broad spectrum antibiotics, a systemic or local use of corticosteroids, administration of neuroleptics, immunosuppressant treatment, chemotherapy or radiotherapy, treatment with a proton pump inhibitor (PPI) or with an H2 antihistamine

In other embodiments, oropharyngeal candidiasis is present or is liable to develop in a patient in an immunocompromised state, in particular an immunocompromised state associated with a disease such as Sjögren syndrome, an HIV infection, uncontrolled diabetes, certain endocrine disorders, malnutrition or malabsorption such as, for example, vitamin B deficiency.

In certain embodiments, the oropharyngeal candidiasis is present in an infant or in an elderly person.

The present invention also relates to the Saccharomyces cerevisiae yeast strain number I-3856, in any form defined above, or the pharmaceutical composition defined above for use in a method for preventing or inhibiting the spread of infection by Candida to the esophagus, the stomach or the small intestine in a patient with oropharyngeal candidiasis.

A more detailed description of certain preferred embodiments of the invention is given below.

### DETAILED DESCRIPTION OF THE INVENTION

As mentioned above, the present invention relates to a Saccharomyces cerevisiae yeast strain that is able to reduce the burden of Candida albicans in the oral cavity and prevent the spread of the infection to other organs of the digestive system, such as the esophagus, the stomach and the small intestine. This strain is therefore useful in the treatment and/or prevention of oropharyngeal candidiasis.

I—Saccharomyces cerevisiae Yeast Strain I-3856

The expression “yeast strain” denotes a relatively homogeneous population of yeast cells. A yeast strain is obtained from a clone, a clone being a population of cells obtained from a single yeast cell.

The Saccharomyces cerevisiae yeast strain used in the context of the present invention is the strain that was deposited on 17 Oct. 2007, by the present applicant, with the CNCM (National Collection of Cultures of Microorganisms, 25 rue du Docteur Roux, 75724 Paris Cedex 15, France) under number I-3856.

This Saccharomyces cerevisiae yeast strain has been described previously, by the present applicant, in document WO 2009/103884, where it is described as being useful in the prevention and/or treatment of pathologies, diseases or disorders of the intestine, including inflammations caused by intestinal colonization by Candida albicans; as well as in document WO 2014/009656 and the article by Cayzeele-Decherf et al. (Med. J. Obst. Gynec., 2017, 5(4): 1112), where it is described as being effective for controlling vaginal proliferation of Candida and for preventing recurrence of vaginal or vulvovaginal candidiasis. It should be noted, however, that the behavior of pathogenic fungi and of probiotics with respect to the oropharyngeal mucosa is not transposable to that adopted with respect to the vaginal mucosa or the intestinal mucosa, the environment of these mucosae in particular being different.

In the context of the present invention, the Saccharomyces cerevisiae strain number I-3856 is in the form of yeast cells obtained by culture (or multiplication) of the starting strain. The culture of a Saccharomyces cerevisiae strain may be carried out by any suitable method. The methods of culture of a Saccharomyces cerevisiae strain are known in the art, and a person skilled in the art knows how to optimize the culture conditions for each strain as a function of its nature. Yeast cells may for example be obtained by multiplication of the Saccharomyces cerevisiae strain number I-3856 in a culture medium as described in the reference work “Yeast Technology”, 2nd Edition, 1991, Reed and Nagodawithana, published by Van Nostrand Reinhold (ISBN 0-442-31892-8).

Thus, for example, on an industrial scale, Saccharomyces cerevisiae yeast cells usable in the context of the present invention may be obtained by a method comprising the following steps:
culture of the Saccharomyces cerevisiae strain number I-3856 in a culture medium in several stages, firstly in semi-anaerobiosis, then in aerobiosis (oxygen-rich medium/atmosphere) to obtain multiplication of the starting yeast cells; and
separation, by centrifugation, of the yeast cells thus produced, to obtain a liquid yeast cream containing between 12% and 25% of yeast dry matter.

The yeast thus obtained is a live yeast. In certain embodiments, the Saccharomyces cerevisiae strain number I-3856 used in the context of the invention is therefore in the form of live yeast. The term “live yeast”, which is synonym of “active yeast”, denotes a population of yeast cells that are metabolically active.

The live yeast strain I-3856 used in the context of the present invention is in the form of dry yeast. Dry yeast is characterized by a low water content, and generally comprises a proportion of yeast dry matter above 90%, preferably a proportion of dry matter between 93% and 96%. One of the advantages of a dry yeast is its long shelf life.

Thus, the method of producing cells of the Saccharomyces cerevisiae yeast number I-3856 may further comprise a subsequent step of drying the cells, to obtain a yeast in the dry form. Said drying may for example be freeze-drying (lyophilization), fluidized bed drying or spray-drying.

In certain embodiments, the dry live yeast strain I-3856 is in the form of an active dry yeast or of an instant dry yeast.

An active dry yeast may be obtained by dehydration of compressed or liquid yeast by the combined action of heat (at low temperature) and mechanical activity, which make it possible to transform a pasty product (compressed or liquid yeast) into a dry product, in the form of spherules. For example, active dry yeast is obtained by extrusion and fluidized-bed drying of a compressed yeast or of a liquid yeast.

An instant dry yeast may be obtained by dehydration of compressed or liquid yeast by the action of a gradient of hot air, which makes it possible to transform a pasty product (compressed or liquid yeast) into fine dry noodles. For it to remain stable, the instant dry yeast must then be packed in the absence of oxygen.

In other embodiments, the Saccharomyces cerevisiae strain number I-3856 used in the context of the present invention is in the form of inactive yeast (as opposed to live yeast). The terms “inactive yeast” and “deactivated yeast”, which are used here indiscriminately, denote a yeast that is no longer alive (therefore a dead yeast), i.e. a yeast whose metabolism has been stopped irreversibly.

An inactive yeast according to the invention may be obtained by any suitable method. Suitable techniques that are familiar to a person skilled in the art include thermal treatment of yeast, a treatment consisting of subjecting yeast to several successive cycles of freezing and thawing, treatment by irradiation, treatment by spraying or any combination of these treatments. An inactive yeast is generally in the dry form.

In another embodiment, it is a derivative or a fraction of cells of Saccharomyces cerevisiae number I-3856 that is used in the context of the present invention, said derivative or said fraction being selected from the group consisting of the walls of said yeast cells, the wall glucans of said yeast cells, the wall mannoproteins of said yeast cells, a filtrate or extract of said yeast cells and any combination thereof.

The terms “yeast cell walls” and “yeast hulls” are used here interchangeably and denote the insoluble fraction of the yeast cells, i.e. the wall and the plasma membrane of yeast.

The terms “yeast filtrate” and “yeast extract” are used here interchangeably, and denote the soluble fraction of the yeast cells, i.e. everything that is not the yeast wall and plasma membrane.

Conventionally, yeast hulls or yeast extracts are obtained by a method comprising a step of autolysis or of enzymatic hydrolysis, essentially by proteases, followed by a step of separating the soluble fraction from the insoluble fraction, the isolated insoluble fraction corresponding to yeast hulls and the soluble fraction corresponding to yeast extract. The insoluble fraction and/or the soluble fraction may then be dried. The method of obtaining yeast hulls is such that it preserves the structural polysaccharides of the cell wall, i.e. the β-glucans and the mannans, these mannans being in the form of mannoproteins. The methods of obtaining yeast hulls and yeast extracts are known in the art (see for example the reference work “Yeast Technology”, 2nd edition, 1991, G. Reed and T. W. Nogodawithana, published by Van Nostrand Reinhold, New York, ISBN 0-442-31892-8).

Yeast hulls may be in the liquid form, in the dry form or in the viscous form. They are considered to be in the dry form when their dry matter content is at least 85%. In contrast, if their dry matter content is less than 20 wt %, they are considered to be in liquid form. Starting from 20 wt % and below 85 wt % of matter dry, yeast hulls are considered to be in the viscous form. The yeast hulls are preferably used in the dry form.

A yeast extract may be in the dry form, preferably in the form of fine water-soluble powder, in the liquid form or in the form of paste. Yeast extract is considered to be in the dry form when its dry matter content is at least 85%. If its dry matter content is below 70 wt %, it is considered to be in the liquid form. Starting from 70 wt % and below 85 wt % of dry matter, yeast extract is considered to be in the form of paste. The yeast extract used is preferably in the dry form, more preferably in the form of fine water-soluble powder. A yeast extract comprises predominantly protein materials, preferably at least 55% of protein materials.

The term “wall β-glucans” denotes the β-glucans of the wall of the yeast cells, which are essentially glucose polymers whose glucose units of the main chain are joined together by β-1,3 bonds and whose branchings are joined together by β-1,6 bonds. The β-glucans of yeast are insoluble and have a low viscosity. A person skilled in the art knows how to extract the β-glucans from the wall of the yeast cells. A common method comprises successive hot extractions with a base and with an acid (such as acetic acid), followed by water washing operations to remove any soluble compounds of the yeast hulls, and recovery of the insoluble material consisting of the wall β-glucans.

The term “wall mannoproteins” denotes yeast polysaccharides and more precisely copolymers of neutral or acid monosaccharides (with 5 or 6 carbon atoms), linked together by glycosidic bonds and bound to proteins. In the yeast walls, the glucans make up a network on which other glucans, chitin, and mannoproteins are bound covalently. The mannoproteins are bound to the glucans via a glycosyl phosphate containing a chain of 5 mannose residues. A person skilled in the art knows how to extract the mannoproteins from the wall of the yeast cells. A common method comprises enzymatic digestion of the yeast walls with a preparation of β-glucanases (for example the industrial product GLUCANEX™), followed by separation of the hydrolyzate by centrifugation, and purification by ultrafiltration. Another method is based on hot chemical extraction.

II—Uses of the Saccharomyces cerevisiae Yeast Strain I-3856 in the Treatment and/or Prevention of Oropharyngeal Candidiasis

The invention therefore relates to the Saccharomyces cerevisiae strain number I-3856 (in any one of the forms described above) for the treatment and/or prevention of oropharyngeal candidiasis. The invention also relates to a method of treatment and/or prevention of oropharyngeal candidiasis in a subject, the method comprising a step of administering an effective amount of the Saccharomyces cerevisiae strain number I-3856 to the subject. The invention also relates to the use of the Saccharomyces cerevisiae strain number I-3856 for making a drug for the treatment and/or prevention of oropharyngeal candidiasis.

In the context of the present invention, “treatment” means a method that has the aim of: (1) delaying or preventing the onset of a disease or a clinical condition; (2) slowing or stopping the progression, aggravation or deterioration of the symptoms of the disease; (3) improving the symptoms of the disease; and/or (4) curing the disease. A treatment may be administered before the start of the disease, for a prophylactic action (it is then called “prevention”), or it may be administered after the onset of the disease, for a therapeutic action.

Here, the term “subject” denotes a mammal, and more particularly a human being, having the possibility of being a victim of an oral infection by Candida, but does not necessarily have oropharyngeal candidiasis. The term “subject” does not denote a particular age and therefore includes neonates, children, adolescents, adults, and the elderly. The term “patient” is sometimes used here, instead of “subject”, when the subject has (i.e. has been diagnosed as having) oropharyngeal candidiasis.

The term “oropharyngeal candidiasis” denotes the lesions of the oral cavity and/or of the oropharynx induced by fungi of the genus Candida, in particular Candida albicans, capable of developing in a host who has become susceptible to infection.

There are several clinical forms of oropharyngeal candidiasis. Some patients will only be affected by a single clinical form, whereas others will be affected by more than one form of candidiasis of the oral mucosae. These forms include:
pseudomembranous buccal candidiasis, better known as thrush, which is characterized by the presence of a sticky whitish substance resembling cottage cheese on the oral mucosa, the patches being characteristically distributed on the buccal mucosa, the palate and on the dorsal surface of the tongue. The patches can be removed by rubbing them with a tongue scraper or with dry gauze. The underlying mucosa then appears normal or erythematous.
erythematous buccal candidiasis, which is characterized by generalized, well defined atrophied erythematous patches or macules. This candidiasis is commoner than pseudomembranous candidiasis;
hyperplasic buccal candidiasis or keratosic candidiasis, which is characterized by whitish patches that do not disappear when scraped;
angular cheilitis or perleche, which is characterized by fissures, desquamation and erythema at the corners of the mouth; and
median rhomboid glossitis, which is characterized by red patches or by red and white patches on the midline of the dorsal surface of the tongue, in front of the circumvallate papillae.

The clinical appearance, often very evocative, is sufficient for a diagnosis of oropharyngeal candidiasis. Biological confirmation by mycological examination is sometimes required in the case of an atypical clinical appearance or in the case of persistent lesions despite appropriate treatment. It is carried out by taking a sample with a swab at the level of the lesion (whitish lesion, erythematous patch, furrows of perlèche). Direct examination of the sample looks for budding pathogens and the presence of pseudo-filaments (pathogenic form). Candida albicans grows in 24-48 hours on specific media, allowing identification of the pathogen with quantification of the number of colonies.

A method of treatment and/or prevention of oropharyngeal candidiasis according to the invention comprises the administration, to a subject, of an effective amount of the Saccharomyces cerevisiae strain number I-3856 (in one of the forms described above). The effective amount, which may be administered in one or more doses, can be determined by the doctor. The exact amount to be administered may vary from one patient to another, depending on age, weight, the patient's general condition, the severity and/or extent of oropharyngeal candidiasis, etc. The effective amount to be administered may also vary depending on the therapeutic effect desired (i.e. prevention of oropharyngeal candidiasis or treatment of oropharyngeal candidiasis). The effective amount to be administered may also vary depending on the chosen route of administration, for example topical or systemic.

For example, a daily dose of yeast I-3856 in the form of live dry yeast may be between 1.10⁷ and 1.10¹¹ CFU, and preferably between 1.10⁹ and 5.10¹⁰ CFU. The term CFU denotes Colony Forming Unit.

For example, a daily dose of yeast I-3856 in the inactive form may be between 1 mg and 10 g, preferably between 100 mg and 5 g.

For example, a daily dose of cell walls of the yeast I-3856 may be between 0.5 mg and 5 g, preferably between 50 mg and 2.5 g.

A method of treatment according to the invention may be used for treating a first episode of oropharyngeal candidiasis or a recurrence. In either case, the patient may previously have been treated with a usual antifungal or antimycotic. Alternatively, the yeast I-3856 may be the first treatment prescribed for the patient.

A method of treatment according to the invention may be used to avoid, reduce, inhibit or prevent the spread of an oral or oropharyngeal infection by Candida to another part of the digestive system such as the esophagus, stomach and small intestine.

A method of treatment according to the invention may also be used for preventing oropharyngeal candidiasis in a patient, whether the candidiasis is a first episode or a recurrence. This may be the case for patients receiving a medical treatment that is known to promote colonization of the oral mucosae by Candida, such as treatment with broad spectrum antibiotics, systemic or local use of corticosteroids, administration of neuroleptics, immunosuppressant treatment, chemotherapy or radiotherapy, treatment with a proton pump inhibitor (PPI) or with an H2 antihistamine This may also be the case for patients in an immunocompromised state, for example associated with a disease such as Sjögren syndrome, an HIV infection, uncontrolled diabetes, certain endocrine disorders, malnutrition or malabsorption such as, for example, vitamin B deficiency. This may also be the case for certain elderly patients.

In certain embodiments, a treatment according to the invention is administered alone. In other words, the yeast I-3856 is the only agent to be administered, apart from optional antiseptic mouthwash.

In other embodiments, a treatment according to the invention is administered in combination with another therapy, for example with the administration of a usual antifungal such as a topical antifungal (for example nystatin as tablets to be sucked or in oral solution or amphotericin B or miconazole in buccal gel) or a systemic antifungal (for example fluconazole or ketoconazole or itraconazole in tablet form).

### III—Pharmaceutical Compositions

As stated above, the Saccharomyces cerevisiae strain number I-3856 may be administered as such (in one of the various forms described above) or in the form of a pharmaceutical preparation or composition in combination with at least one physiologically acceptable excipient. Thus, more specifically, a pharmaceutical composition according to the invention comprises an effective amount of the Saccharomyces cerevisiae yeast strain number I-3856 and at least one physiologically acceptable excipient. A pharmaceutical composition according to the invention may be classified as a pharmaceutical preparation available on prescription or over the counter.

In the context of the present invention, “physiologically acceptable excipient” means any medium or additive that does not interfere with the efficacy of the biological activity of the active pharmaceutical ingredient (here, the Saccharomyces cerevisiae strain), and that is not excessively toxic for the patient or subject, at the concentrations at which it is administered. A physiologically acceptable excipient may be an excipient suitable for administration to mammals, in particular to humans.

The pharmaceutical compositions according to the present invention may be administered using any combination of dosage and route of administration that is effective for obtaining the desired therapeutic/prophylactic effect. As already stated above, the exact amount to be administered may vary from one patient to another, depending on age, weight, the patient's general condition, the severity and extent of the Candida infection, etc. The route of administration (topical or systemic) may be selected as a function of the severity and extent of the Candida infection and/or as a function of the patient's age and/or health.

As an example, the invention relates to a pharmaceutical composition as defined above for daily use of yeast I-3856 in the form of live dry yeast in an amount from 1.10⁷ CFU to 1.10¹¹ CFU, preferably in an amount from 1.10⁹ CFU to 5.10¹⁰ CFU, and the effective daily dose may be administered in one, two or three doses.

As an example, the invention relates to a pharmaceutical composition as defined above for daily use of yeast I-3856 in the inactive form in an amount between 1 mg and 10 g, preferably between 100 mg and 5 g, and the effective daily dose may be administered in one, two or three doses.

As an example, the invention relates to a pharmaceutical composition as defined above for daily use of cell walls of the yeast I-3856 in an amount between 0.5 mg and 5 g, preferably between 50 mg and 2.5 g, and the daily dose may be administered in one, two or three doses.

The formulation of a pharmaceutical composition according to the present invention may vary as a function of the route of administration and of the dosage for which the composition is intended to be used. After formulation with at least one physiologically acceptable excipient, a pharmaceutical composition of the invention may be in any form suitable for administration to a mammal, in particular a human, for example in the form of lozenges, tablets, sugar-coated pills, capsules, sirup pearls, emulsions, ointments, pastes, gels, powders, sachets, injectable solutions, etc. A person skilled in the art is able to select the most suitable vehicles and excipients for preparing a given type of formulation. A composition according to the invention may further comprise additives such as preservatives, sweeteners, flavorings, thickening agents, colorants, humectants, disintegrants, absorption accelerators, lubricants, etc.

In certain embodiments, a pharmaceutical composition according to the invention only contains a single active agent: the yeast I-3856 (or the cell walls). Said pharmaceutical composition does not contain, in particular, another probiotic or a combination of probiotics.

In other embodiments, a pharmaceutical composition according to the invention additionally contains at least one additional active pharmaceutical ingredient (i.e., in addition to the yeast I-3856 or the cell walls). “Active pharmaceutical ingredient” means any compound or substance whose administration has a therapeutic effect or whose administration has a beneficial effect on the health or the general condition of a patient or of a subject to whom or to which it is administered.

Thus, an active pharmaceutical ingredient may be active against infection of the oral mucosae by Candida that we wish to prevent or treat by administration of the pharmaceutical composition; or may be active against a condition or a symptom associated with oropharyngeal candidiasis (for example, pains or fever); or else may increase the availability and/or the activity of the active ingredient or ingredients of the pharmaceutical composition.

Examples of active pharmaceutical ingredients that may be present in a composition of the present invention include, without limitation, active ingredients having a soothing, anti-irritant, analgesic, anti-inflammatory, wound-healing, antibiotic, antipyretic, or antifungal activity (without any effect on the yeast I-3856), etc.

Unless defined otherwise, all the technical and scientific terms used in the description have the same meaning as that commonly understood by a person of average skill in the field to which this invention belongs. Moreover, all the publications, patent applications, patents and all other references mentioned here are incorporated by reference.

### EXAMPLES

The following examples describe certain embodiments of the present invention. However, it is to be understood that the examples and the figures are only presented for purposes of illustration and do not in any way limit the scope of the invention.

### LEGEND OF THE FIGURES

FIG. 1: Quantification of the total photon flux emission from the oral cavity of mice infected by BLI Candida albicans and treated with different compounds, measured on days +1, +3 and +6 after infection. The results give the mean value±SEM of 5 mice per group in 3 different experiments. ≠, p<0.05 (infected mice treated with FLU, GI or IY vs. infected mice treated with saline solution).

FIG. 2: Imaging of mice in vivo infected by BLI Candida albicans and treated with different compounds, carried out on day +6 after infection.

FIG. 3: CFU count on the tongue of mice infected by Candida albicans and treated with different compounds, carried out on days +3 and +6 after infection. The results give the mean value±SEM of 3 mice per group in 3 different experiments. ≠, p<0.05 (infected mice treated with FLU, GI or IY vs. infected mice treated with saline solution).

FIG. 4: Histological examination on day +8 (A) carried out on uninfected mice treated with different compounds and (B) carried out on mice infected by BLI Candida albicans and treated with different compounds.

FIG. 5: CFU count (A) in the esophagus, (B) in the stomach, and (C) in the duodenum, of mice infected by BLI Candida albicans and treated with different compounds, carried out on day +6 after infection. The results give the mean value±SEM of 3 mice per group in 3 different experiments. ≠, p<0.05 (infected mice treated with FLU, GI or IY vs. infected mice treated with saline solution).

FIG. 6: Ex vivo imaging of the esophagus and stomach of mice infected by BLI Candida albicans and treated with different compounds, carried out (A) on day +6 and (B) on day +8 after infection. The results give the mean value±SEM of 5 mice per group in 3 different experiments. ≠, p<0.05 (infected mice treated with FLU, GI or IY vs. infected mice treated with saline solution).

FIG. 7: CFU counts on the tongue. The fungal burden of the tongue of the infected mice treated with 10 μl of saline solution, or of fluconazole (FLZ—4 mg/ml), or of IY, GI or WG (all at 100 mg/ml on days +1, +2 and +3 post-infection, was evaluated by a CFU assay on days +1, +3 and +6 after infection of the tongue. The results give the mean value ±SEM of 4 to 6 mice in 2 different experiments. #, p<0.05 (infected mice treated with FLZ, IY, GI or WG vs. infected mice treated with saline solution).

FIG. 8: Effects of IY, GI and WG on expression of SAP2, SAP6, ALS3 and HWP1 during oropharyngeal candidiasis. The expression of the genes (A) SAP2, (B) SAP6, (C) ALS3 and (D) HWP1 was analyzed in cellular fractions of homogenates of tongues of infected mice treated with 10 μl of saline solution, or of fluconazole (FLZ—4 mg/ml), or of IY, GI or WG (all at 100 mg/ml) on days +1, +2 and +3 post-infection. On days 3+ and +6 post-infection, the tongue homogenates were centrifuged, then the cellular fractions were lysed and the total RNA was extracted and reverse-transcribed into cDNA. The genes SAP2, SAP6, ALS3 and HWP1 of Candida albicans were detected by real-time PCR and the amounts of cDNA were reported in 2^{-ΔΔCT} relative to the transcripts of the Candida albicans inoculum. The results give the mean value±SEM of samples in triplicate from 4 to 6 mice in 2 different experiments. #, p<0.05 (infected mice treated with FLZ, IY, GI or WG vs. infected mice treated with saline solution).

FIG. 9: Destructive Activity of the Peritoneal Neutrophils. The destructive activity of the peritoneal neutrophils of uninfected mice or of infected mice treated with 10 μl of saline solution, or of fluconazole (FLZ—4 mg/ml), or of IY, GI or WG (all at 100 mg/ml) on days +1, +2 and +3 post-infection, was evaluated on days 3+ and +6 post-infection. The peritoneal neutrophils (1×10⁶/ml) were incubated in the presence of Candida albicans (CA-6) (1×10⁵/ml) for 2 hours. The results give the mean value±SEM for 4 to 6 mice from each group in 2 different experiments. #, p<0.05 (infected mice treated with FLZ, IY, GI or WG vs. infected mice treated with saline solution).

FIG. 10: Production of IL-1α, TNF-α and IL-6. The supernatants of tongue homogenates of uninfected mice or of infected mice treated with 10 μl of saline solution, or of fluconazole (FLZ—4 mg/ml), or of IY, GI or WG (all at 100 mg/ml) on days +1, +2 and +3 post-infection were tested, by ELISA, for the presence (A) of IL-1β, (B) of TNF-α and (C) of IL-6, on days +1, +3 and +6 post-infection. The results give the mean value±SEM for 4 to 6 mice from each group in 2 different experiments. *, p<0.05 (infected mice treated with saline solution vs uninfected mice). #, p<0.05 (infected mice treated with FLZ, IY, GI or WG vs. infected mice treated with saline solution).

FIG. 11: Production of IL-17A/F, IL-22 and IL-23. The supernatants of tongue homogenates of uninfected mice or of infected mice treated with 10 μl of saline solution, or of fluconazole (FLZ—4 mg/ml), or of IY, GI or WG (all at 100 mg/ml) on days +1, +2 and +3 post-infection were tested, by ELISA, for the presence (A) of IL-17A/F, (B) of IL-22 and (C) of IL-23, on days +1, +3 and +6 post-infection. The results give the mean value±SEM for 4 to 6 mice from each group in 2 different experiments. *, p<0.05 (infected mice treated with saline solution vs uninfected mice). #, p<0.05 (infected mice treated with FLZ, IY, GI or WG vs. infected mice treated with saline solution).

FIG. 12: Production of IFN-α. The supernatants of tongue homogenates of uninfected mice or of infected mice treated with 10 μl of saline solution, or of fluconazole (FLZ—4 mg/ml), or of IY, GI or WG (all at 100 mg/ml) on days +1, +2 and +3 post-infection were tested, by ELISA, for the presence of IFN-α on days +1, +3 and +6 post-infection. The results give the mean value±SEM for 4 to 6 mice from each group in 2 different experiments. *, p<0.05 (infected mice treated with saline solution vs uninfected mice). #, p<0.05 (infected mice treated with FLZ, IY, GI or WG vs. infected mice treated with saline solution).

### Example 1

### Evaluation of the Activity of Different Yeast Products in an Animal Model of Oropharyngeal Candidiasis

### A. Materials and Methods

Animal model of oropharyngeal candidiasis. As Candida albicans is not a commensal species in laboratory mice, the procedure developed by Solis and Filler (Nature Protoc., 2012, 7(4): 637-642) for obtaining a reproducible infection that mimics pseusomembranous oropharyngeal candidiasis in humans was used on wild-type C57BL/6 mice. The procedure comprises the injection of cortisone acetate, which makes mice susceptible to oral infection by Candida and infection by Candida albicans.

More specifically, female C57BL/6 mice (Charles River, Calco, Italy), aged from 6 to 8 weeks, were kept in the animal house of Perugia University (Italy). The mice were treated with 225 mg/kg of cortisone acetate (Sigma-Aldrich) every other day beginning one day before infection and were then infected with a suspension of 1×10⁶/ml BLI Candida albicans as described previously (Solis and Filler, Nature Protoc., 20125, 7: 637-642) under anesthesia with subcutaneous injection of a mixture of Tiletamine/Zolazepam-Xylazine (50 mg/kg/5 mg/kg) (Mosci et al., Virulence, 2013, 4: 250-254). The oral cavity was tested immediately before infection to confirm prior absence of Candida species (by taking a buccal sample, which was spread on YPD agar with chloramphenicol (50 μg/ml) (both from Sigma-Aldrich)).

The mice were used in conditions free from specific pathogens—conditions that were checked with tests of sensitivity to undesirable infections. According to the standards of the Federation of European Laboratory Animal Science Associations, no infection was detected. The procedures involving the animals and their care were conducted in accordance with national and international laws and standards. All the experiments on the animals were conducted in compliance with European Directive 2010/63, the European Convention on the Protection of Vertebrates used for Experimental Purposes or for other Scientific Purposes and the national law 116/92. The protocol was approved by the ethics committee of Perugia University for the care and use of animals. All the animals were kept in the animal house of Perugia University. The mice were acclimated for 1 week before beginning the experiments. Each cage contained at most 5 mice, which received food and water ad libitum.

Products tested. The Saccharomyces cerevisiae strain I-3856 in the live form (GI) and in the inactivated form (IY) were tested in this example. After infection, the mice received an oral injection (10 μl) of saline solution (0.9% NaCl, negative control), of fluconazole (FLZ—reference antifungal used as positive control) (4 mg/ml) or GI and IY yeast products (both at 100 mg/ml) on days +1, +2, +3 and +6.

Candida. The strain of Candida albicans CA1398 bearing the fusion product ACT1p-gLUC59 (gLUC59) was used. Culture of C. albicans was maintained over several passages on YPD agar (Y: yeast extract, P: peptone and D: anhydrous dextrose—all from Sigma-Aldrich). The fungal cells were collected by suspending a single colony of Candida albicans in a saline solution, washed twice, counted using a hemocytometer, and adjusted to the required concentrations.

Evaluation of Infection by C. albicans in the Oral Cavity.

a. CFU assay. The number of colonies of Candida albicans adhering to the oral cavity was evaluated on days +3 and +6 after infection with Candida albicans by spreading dilutions of tongue homogenates on CHROMAgar™ plates (growth medium specific and selective for Candida). Then the viable colonies of Candida albicans were counted after two days of culture at 30° C. The results were expressed in Log CFU/g tissue (or Log UFC/g in French).

b. Imaging of BLI Candida in the oral cavity. On days +1, +3 and +6 after infection, the mice received 10 μl (0.5 mg/ml in a 1/10 methanol/H₂O mixture) of coelenterazine (Synchem, OHM), and were then imaged using an IVIS-200™ system (Xenogen Inc.) under anesthesia with 2.5% of isoflurane. On the images, the total emission of photon flux from the oral cavity (Region of Interest, ROI) of each mouse was quantified using Living ImageR software. No luminescence was observed for the uninfected mice that received 10 μl of coelenterazine (data not shown).

c. Histological Examination. Histological examination of the tongue of the mice was carried out on day +8 after infection according to the protocol described by Mosci et al., Virulence, 2013, 4(3): 250-254.

Spread of Infection to the Esophagus, Stomach and Intestine.

a. CFU assay. The number of colonies of Candida albicans that developed, after spread of the infection, in the esophagus, stomach and duodenum, was evaluated on day +6 after infection with Candida albicans by spreading dilutions of homogenates of the tissues/organs on CHROMAgar™ plates. The results were expressed in Log CFU/g tissue (or Log UFC/g in French).

b. Imaging of BLI Candida. On days +6 and +8 after infection, the gastric tracts were excised and 10 μl (0.5 mg/ml in a 1/10 methanol/H₂O mixture) of coelenteratine (Synchem, OHM) was injected via the pharynx into the lumen of the esophagus. The esophagus and the stomach of the mice were then imaged ex vivo using an IVIS-200™ system (Xenogen Inc.). On the images, the total emission of photon flux (Region of Interest, ROI) for each esophagus/stomach system of each mouse was quantified using Living ImageR software.

Statistical analyses. The differences between the infected mice treated with FLU, GI or IY and the infected mice treated with saline solution were evaluated with the Mann-Whitney U test. A value of p<0.05 was regarded as significant.

B. Results

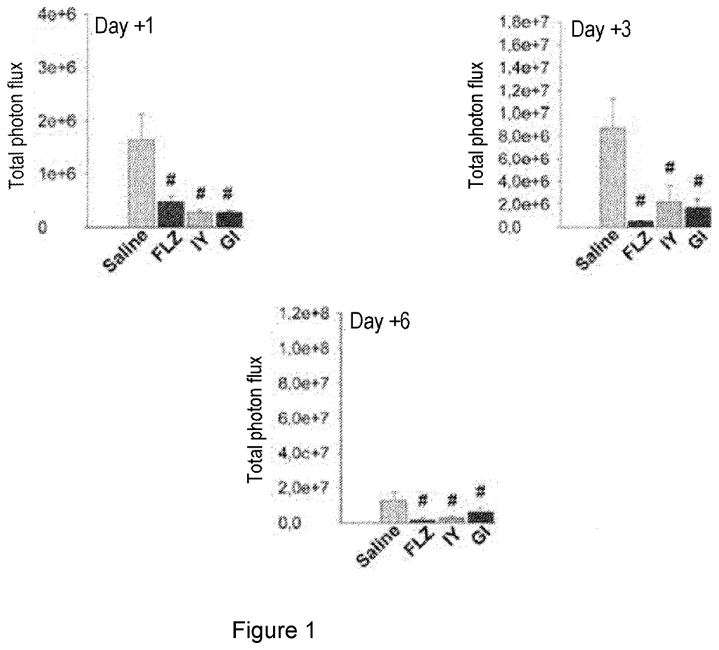

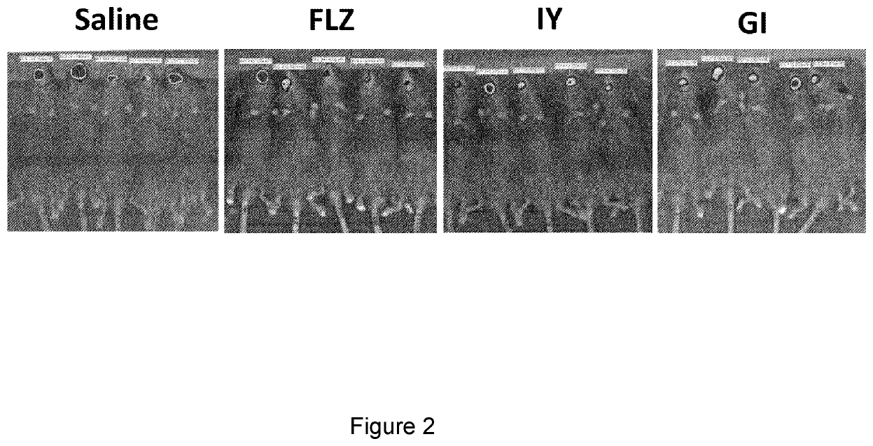

Evaluation of infection by Candida albicans in the oral cavity. FIG. 1 and FIG. 2 show the results of the in vivo imaging of the infected mice treated with the various compounds. These results show that the Saccharomyces cerevisiae strain CNCM I-3856 both in its live form (GI) and in its dead form (IY) is capable of reducing the fungal burden considerably. The beneficial effect is similar to that obtained using fluconazole (FLU—positive control).

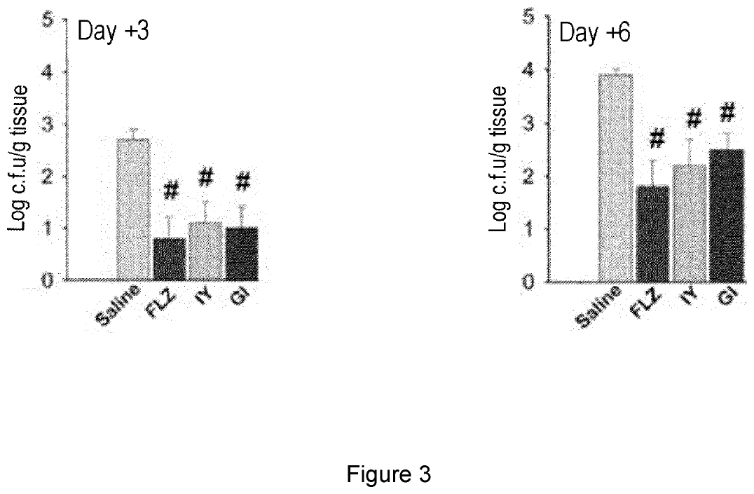

The number of colonies of Candida albicans adhering to the tongue of the mice evaluated on days +3 and +6 after infection with Candida albicans confirms that the Saccharomyces cerevisiae strain CNCM I-3856 both in its live form (GI) and in its dead form (IY) is capable of reducing the fungal burden significantly. The beneficial effect is similar to that obtained using fluconazole (FLU—positive control). (FIG. 3).

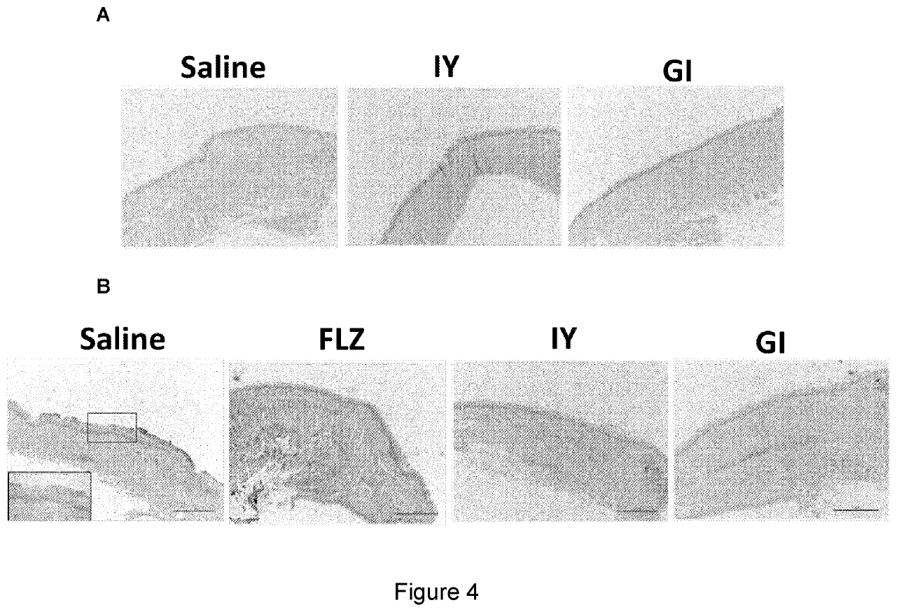

Histological examination carried out on uninfected mice treated with the two yeast products (FIG. 4(A)) shows that the tongue of the uninfected mice treated with GI and IY does not have any lesion and that no recruitment of neutrophils was observed. Histological examination carried out on infected mice treated with the various compounds (FIG. 4(B)) demonstrated that the tongue of the infected mice treated with the Saccharomyces cerevisiae strain CNCM I-3856 both in its live form (GI) and in its dead form (IY) does not have any lesion induced by Candida albicans. The beneficial effect is similar to that obtained using fluconazole (FLU—positive control).

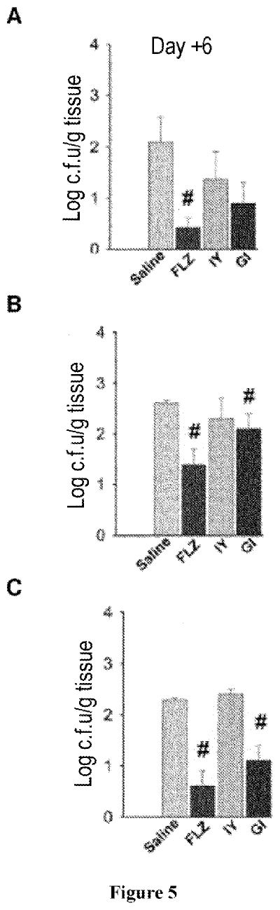

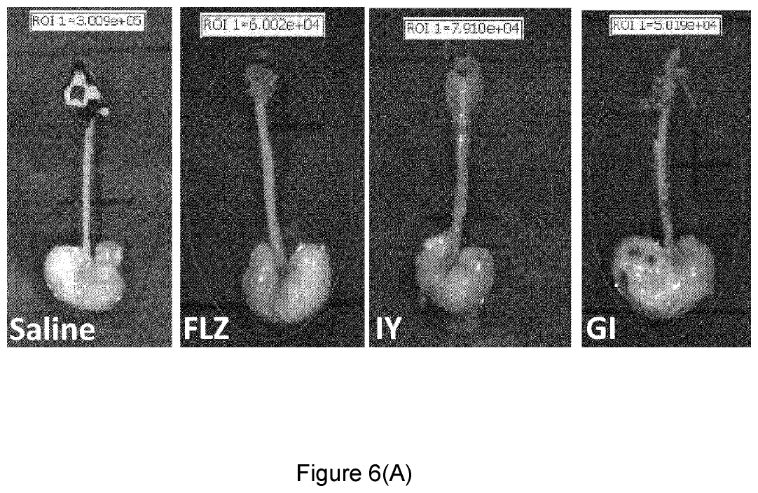

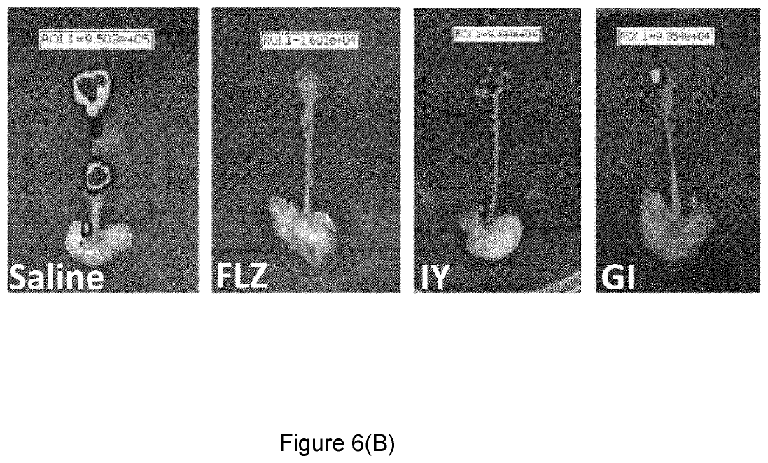

Spread of infection to the esophagus, stomach and duodenum. Determination of the number of colonies of Candida albicans that developed, after spread of the infection, in the esophagus (FIG. 5(A)), in the stomach (FIG. 5(B)) and in the duodenum (FIG. 5(C)) was evaluated on day +6 after infection with Candida albicans. The results obtained show that the Saccharomyces cerevisiae strain CNCM I-3856 in its live form (GI) is capable of inhibiting the fungal load significantly whereas its dead form (IY) shows a tendency to reduce the fungal load in the esophagus and the stomach. The beneficial effect of GI is similar to that obtained using fluconazole (FLU—positive control). Ex vivo imaging of the esophagus and stomach on days +6 (FIG. 6A)) and +8 (FIG. 6(B)) after infection confirms that the Saccharomyces cerevisiae strain CNCM I-3856 in its live form (GI) and to a lesser degree in its dead form (IY) prevents spread of the infection in the digestive tract.

C. Conclusions

Considered collectively, the results obtained in Example 1 show that the Saccharomyces cerevisiae strain CNCM I-3856 both in its live form (GI) and in its dead form (IY) is capable of reducing the fungal burden considerably, thus preventing its spread to the esophagus and stomach. The beneficial effects are similar to those obtained using fluconazole (positive control).

### Example 2

### Effects of the Saccharomyces Cerevisiae Strain I-3856 on the Immune Response to Oropharyngeal Infection by Candida albicans

Saliva is one of the innate defense mechanisms against oral infection by Candida. Saliva forms a film on the teeth and the oral epithelium. The main components of this film are mucins and immunoglobulin A (IgA), which may aggregate Candida albicans, which is removed by the action of swallowing. Moreover, saliva contains bioactive agents, such as histatin-5, lysozyme, lactoferrin and calprotectin having fungicidal properties. Although these agents are present in the saliva at low concentrations, their combined effects are either additive or synergistic. Furthermore, the combination of the salivary defense agents and the dynamic effects of the flow of saliva limit the colonization, proliferation and invasion of the oral epithelium by Candida albicans, which leads to innate oral resistance to Candida albicans (Feller et al., J. Oral. Pathol. Med., 2014, 43: 563-569).

The neutrophils and the T cells play an important role in anti-Candida mucosal immunity. The neutrophils phagocytize and kill the Candida cells by oxidative mechanisms (Moyes et al., Clin. Dev. Immunol., 2011, 346307). The T cells that are mainly involved in the oral response to Candida are the helper T lymphocytes (helper T cells, Th) Th1 and Th17. The Th1 cells produce IFN-γ, which is an effective activator of the neutrophils (Gattoni et al., Clin. Ter., 2006, 157: 457-468). In the presence of IL-6, IL-1β and TGF-β, the T cells differentiate into Th17 and undergo maturation by stimulation with IL-23. The Th17 cells produce IL-17A, IL-17F and IL-22. IL-17A and IL-17F stimulate the epithelial cells to produce antimicrobial peptides and promote recruitment and activation of the neutrophils, thus allowing fungal elimination. IL-22 has effects similar to those of IL-17 with respect to epithelial cells and limits fungal growth (Hebecker et al., Expert. Rev. Anti Infect. Ther., 2014, 12: 867-879; Moyes et al., Clin. Dev. Immunol., 2011, 346307).

It was demonstrated in Example 1 that the Saccharomyces cerevisiae strain number I-3856, whether in the live form (GI) or in the inactivated form (IY), is able to reduce the oral fungal burden, preventing spread of Candida infection to the esophagus and the stomach. In the present example, the ability of WG (cell walls of the Saccharomyces cerevisiae strain CNCM I-3856) to reduce the oral fungal burden was first evaluated, and then it was determined whether oral administration of IY, GI and WG is able to influence the fungal virulence factors and the inflammatory response in oropharyngeal candidiasis.

A. Materials and Methods

Strain of Candida albicans and Culture Conditions. The highly virulent Candida albicans strain (CA-6) was used (Bistoni et al., Infect. Immun., 1986, 51: 6668-674). The Candida albicans culture was maintained by successive passages on YPD agar (Y: yeast extract, P: peptone and D: anhydrous dextrose—all from Sigma-Aldrich). The fungal cells were collected by suspending a single colony of Candida albicans in a saline solution, washed twice, counted using a hemocytometer and adjusted to the required concentrations.

Animal model of Oropharyngeal candidiasis. Female C57BL/6 mice (Charles River, Calco, Italy), aged from 6 to 8 weeks, were kept in the animal house of Perugia University (Italy). The mice were treated with 225 mg/kg of cortisone acetate (Sigma-Aldrich) every other day beginning one day before infection and were then infected with a suspension of 1×10⁶/ml Candida albicans (CA-6) as described previously (Solis and Filler, Nature Protoc., 20125, 7: 637-642) under anesthesia with subcutaneous injection of a mixture of Tiletamine/Zolazepam-Xylazine (50 mg/kg/5 mg/kg) (Mosci et al., Virulence, 2013, 4: 250-254). The oral cavity was tested immediately before infection to confirm prior absence of Candida species (by taking a buccal sample, which was spread on YPD agar with chloramphenicol (50 μg/ml) (both from Sigma-Aldrich). See Example 1 for the ethical declarations.

Products tested. In this example, the Saccharomyces cerevisiae strain I-3856 was tested in the live form (GI) and in the inactivated form (IY) but also in the form of cell walls (WG). After infection, the mice received an oral injection (10 μl) of saline solution (0.9% NaCl, negative control), of fluconazole (FLZ, 4 mg/ml, positive control) and of IY, GI or WG (all at 100 mg/ml) on days +1, +2 and +3 post-infection.

CFU assay. See Example 1 for the operating conditions. The fungal burden was determined on days +1, +3 and +6 post-infection.

Quantitative Analysis of Expression of the SAP2, SAP6, ALS3 and HWP1 genes. Homogenates of tongues from mice with oral infection by Candida albicans and treated as described above with saline solution, or fluconazole, or with IY, GI or WG, were obtained on days +1, +3 and +6 post-infection. The mouse tongue homogenates were centrifuged at 3000 rev/min for 5 minutes, and then the cellular fractions were lysed with Trizol (Life Technology).

The total RNA was extracted and reverse-transcribed using the reverse transcriptase reaction of the Moloney murine leukemia virus (M-MLV RT) according to the manufacturer's instructions. The concentration of complementary DNA (cDNA) was determined with a spectrophotometer. The SAP2, SAP6, ALS3 and HWP1 genes of Candida albicans were detected using known primers (Naglik et al., J. Med. Microbiol., 2006, 55: 1323-1327; Naglik et al., Microbiology, 2008, 154: 3266-3280; Roudbarmohammadi et al., Adv. Biomed. Res., 2016, 5: 105). The real-time PCR reactions were carried out in 96-well PCR plates with SYBR green (BioRad). For the real-time PCR reactions, 200 ng of cDNA was used. All the samples were measured in triplicate. The relative expression levels of the Candida genes at different times post-infection were reported in 2^{-ΔΔCT} relative to the transcripts of the Candida albicans inoculum (Pericolini et al., Virulence, 2017, 8: 74-90)). The amplification conditions used are the same for SAP2, SAP6, ALS3 and HWP1: 3 minutes at 95° C., 40 cycles of 10 seconds at 95° C. and 30 seconds at the specific hybridization temperature of the primer. The experiments were carried out with an Eppendorf Mastercycler.

Tests of Candidacidal Power. On days +3 and +6 post-infection, the peritoneal neutrophils of uninfected mice and of mice infected and treated as described above, were collected 18 hours after an intraperitoneal injection of 0.5 ml of solution of 10% thioglycolate without endotoxin (Difco).

The neutrophil destruction activity was determined by a CFU inhibition assay. Briefly, neutrophils (10⁵ cells) in a suspension of 0.1 ml per well were incubated in a flat-bottomed 96-well tissue culture plate with 10⁴ cells of Candida albicans (CA-6) in 0.1 ml of RPMI containing 5% of FBS and were incubated for 2 hours at 37° C., in the presence of 5% CO₂. After incubation, the plates were agitated vigorously and the cells were lysed by adding Triton X-100 (0.1% in distilled water, final concentration of 0.01% in each well). Serial dilutions were prepared from each well with distilled water. The samples were spread on Sabouraud dextrose agar with chloramphenicol (50 mg/ml) in triplicate and the CFU values were evaluated after incubation for 24 hours at 37° C. The so-called control cultures consisted of Candida albicans (CA-6) incubated with RPMI-1640 containing 5% of FCS without effector cells.

Production of Cytokines. Tongue homogenates, from uninfected mice or from mice orally infected and treated as described above, were obtained on days +1, +3 and +6 post-infection. The tongue homogenates were centrifuged at 3000 rev/min for 5 minutes and the supernatants were collected and the levels of IL-1β, TNF-α, IL-6, IL-17A/F, IL-22, IL-23 and IFN-γ were measured by ELISA assays (eBioscience).

Statistical analyses. The results are the mean values±SEM of samples in duplicate or triplicate from 4 to 6 mice for each group in two different experiments. The differences between the infected mice treated with saline solution and the uninfected mice or the infected mice treated with FLU, GI, IY or WG and the infected mice treated with saline solution were evaluated with the Mann-Whitney U test. A value of p<0.05 was regarded as significant.

B. Results and Discussion

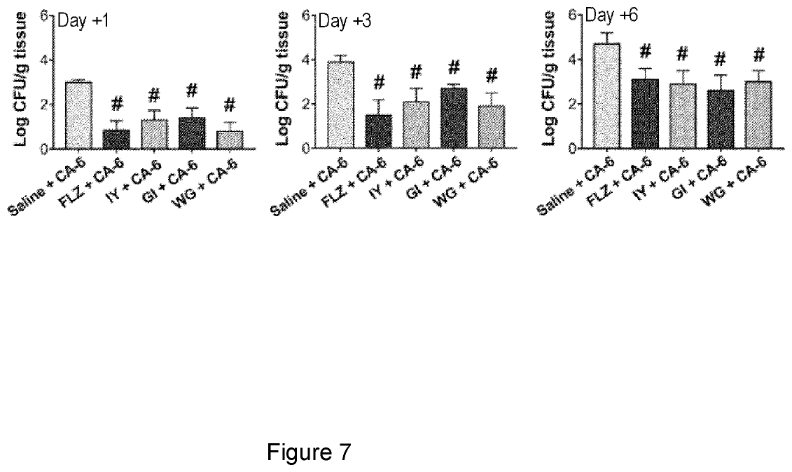

The capacity of WG (cell walls of the Saccharomyces cerevisiae strain CNCM I-3856) was first evaluated for its influence on the burden of Candida albicans in the oral cavity. For this purpose, the mice were treated with IY, GI or WG (all at 100 mg/ml) on days +1, +2 and +3 after infection with Candida albicans (10⁶/ml). The CFU values of the tongues of the mice thus treated were evaluated on days +1, +3 and +6 after infection. The results obtained, which are presented in FIG. 7, show that all the compounds tested, including WG, are capable of significantly inhibiting the Candida albicans burden, and the beneficial effect is similar to that obtained using fluconazole (FLZ).

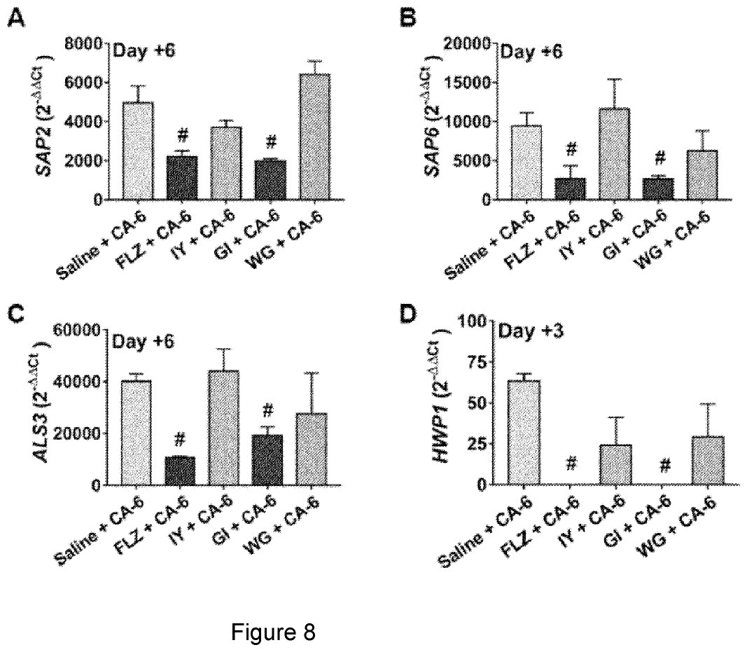

Next, the present inventors determined whether inhibition of the fungal burden was linked to inhibition of certain virulence factors of Candida albicans. Therefore they determined the expression of aspartic proteases (Sap), which are involved in invasion of the tissues by Candida. Determination of the SAP2 and SAP6 genes was carried out on days +1, +3 and +6 post-infection. The results, presented in FIGS. 8(A) and (B), show that there is significant inhibition of the expression of SAP2 and SAP6 after treatment with GI but not with IY or with WG—the effect being observed 6 days after infection.

Candida albicans expresses the invasin Als3, which binds to the epithelial cells, resulting in endocytosis of the fungus by invasion and active penetration of the epithelial cells producing cell damage and release of pro-inflammatory cytokines (Naglik et al., Microbes Infect., 2011, 13: 963-976). The HWP1 (hyphal wall protein 1) gene of Candida albicans codes for a protein of the fungal cell wall that is necessary for hyphal growth and for adhesion of the fungus to the epithelial cells (Orsi et al., Microb. Pathog., 2014, 69-70: 20-27). Expression of the two genes ASL3 and HWP1 was found to be reduced significantly after treatment with GI but not with IY or with WG (see FIGS. 8(C) and (D)). These results suggest that the inhibition of CFU by GI could be due to inhibition of adhesion of Candida to the epithelial cells as well as to inhibition of the yeast-hyphal transition with reduced consequent cell damage. Since IY and WG do not affect gene expression in Candida, there are other mechanisms of action responsible for the reduction of CFU observed with these two compounds.

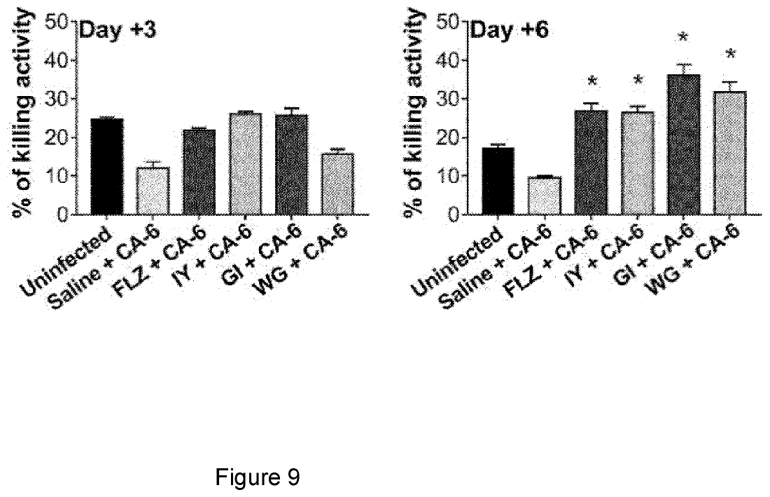

The neutrophils are major effector immune cells in fungal destruction (Gazendam et al., Immunol. Rev., 2016, 273: 299-311). Therefore the destructive activity of the neutrophils was examined. The results obtained are presented in FIG. 9. They show that the destructive activity of the neutrophils, which is weakened during infection with Candida albicans relative to the uninfected mice, is restored by treatment with the yeast products tested. In particular, 6 days after infection, a large increase in the destructive activity of the neutrophils was observed in the infected mice treated with GI, IY and WG compared to the untreated infected mice.

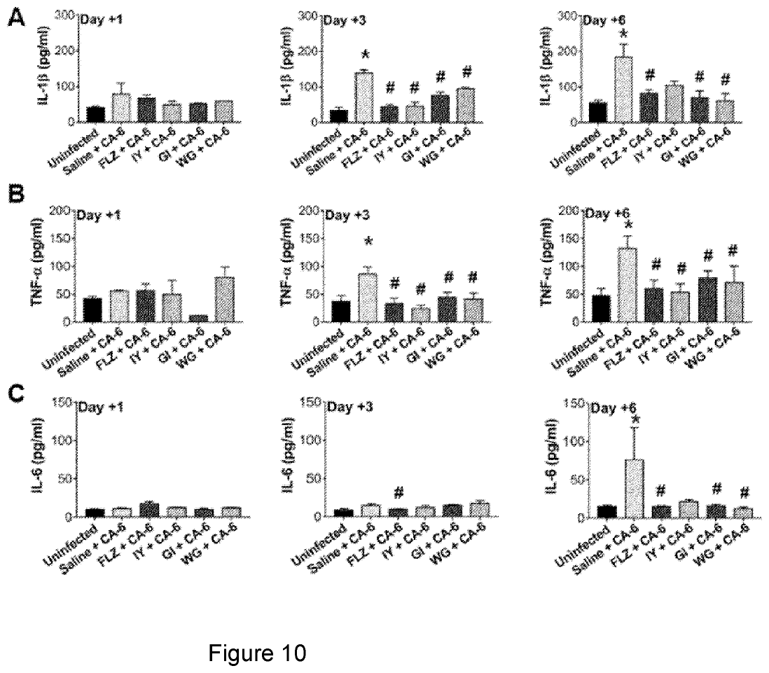

In the course of oral candidiasis, by releasing pro-inflammatory cytokines the epithelial cells induce recruitment of neutrophils, which limit the extent of the damage to the epithelial cells by accelerating elimination of Candida (Trautwein-Weidner et al., Mucosal. Immunol., 2015, 8: 221-231). Production of the pro-inflammatory cytokines IL-1β, TNF-α and IL-6 was tested in the experimental system used here. The results obtained are presented in FIG. 10. These results show that one day after infection, no change in the production of the pro-inflammatory cytokines, such as IL-1β, TNF-α and IL-6, was observed in the infected mice, whether or not treated with the various compounds, compared to the uninfected mice. However, 3 days after infection, the infected mice display a significant increase in production of IL-1β and TNF-α, and treatment with the yeast products or with FLZ results in considerable inhibition of these cytokines. At this moment, the production of IL-6 remains unchanged compared to the untreated mice with the exception of FLZ. When analysis of the pro-inflammatory cytokines was carried out 6 days post-infection, a large increase was observed in the supernatants of tongues of infected mice compared to the supernatants of tongues of uninfected mice, and treatment with GI, WG and FLZ led to a significant decrease in the production of all the cytokines, whereas IY was only capable of significantly inhibiting the production of TNF-α (FIG. 10).

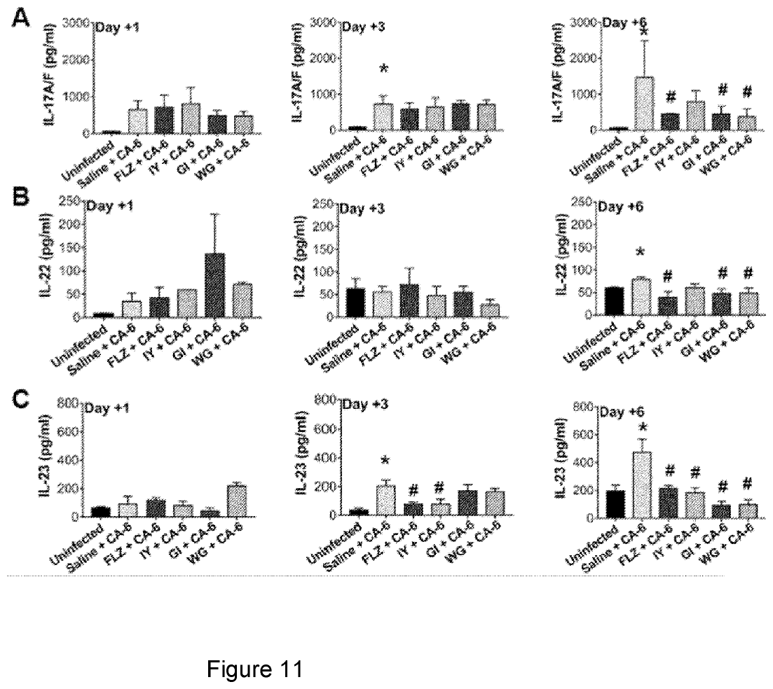

It is known that IL-17, IL-22 and IL-23 play a role in the elimination of the fungi during oral candidiasis (Hebecker et al., Expert. Rev. Anti Infect. Ther., 2014, 12: 867-879; Moyes et al., Clin. Dev. Immunol., 2011, 346307). Therefore the presence of the cytokines IL-17, IL-22 and IL-23 of the T cells was also determined in the present system. The results obtained (FIG. 11) show that, 6 days after infection, the presence of all these cytokines is greater in the supernatants of tongues of the infected mice than in the supernatants of tongues of the uninfected mice. Treatment with GI and WG resulted in significant inhibition of all the cytokines tested, whereas IY only induced a significant decrease in the case of the production of IL-23.

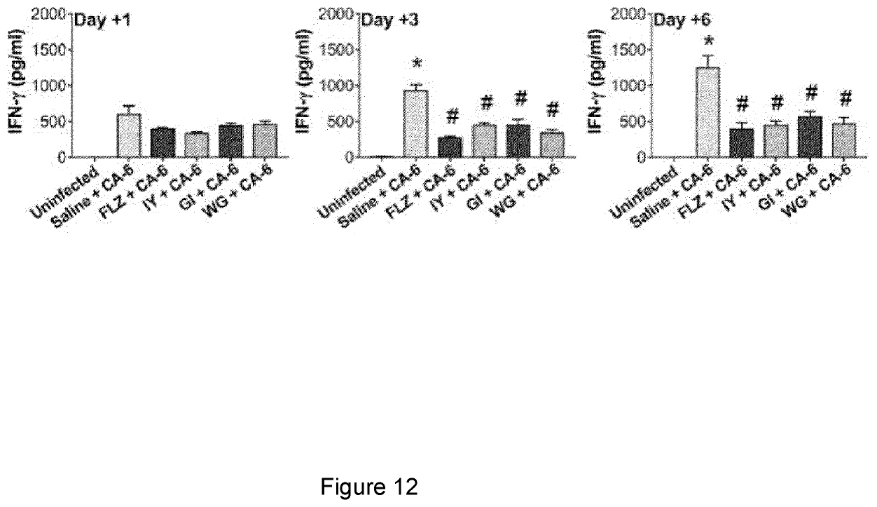

An increase in IFN-γ, a cytokine typical of the T cells, was also observed in the supernatants of tongues of infected mice, 3 days and 6 days post-infection, and treatment with the test compounds led to a large reduction in the production of IFN-γ (see FIG. 12).

Considered together, these results suggest that the inhibition of the pro-inflammatory cytokines observed after treatment with the test compounds could be attributed to a decrease in the fungal burden. It is conceivable that by inhibiting several virulence factors, such as the aspartic proteases and the adhesins of Candida, GI succeeds in inhibiting the Candida burden and the inflammatory response. In the experiments presented here, IY and WG apparently do not have an effect on the virulence factors of Candida, but they induce a large reduction in the fungal burden. It has previously been shown that IY is capable of producing strong aggregation with Candida (Pericolini et al., Virulence, 2017, 8: 74-90). It is conceivable that WG is effective by a similar mechanism. Moreover, the increase in the destructive activity of the neutrophils observed after treatment with all the compounds tested could also explain the large decrease in the fungal burden.

C. Conclusions

The results obtained demonstrate that all the yeast products tested (GI, IY and WG) are capable of inhibiting fungal growth of Candida in the oral cavity. The beneficial effect of GI is at least partly due to inhibition of the adhesion of Candida to the epithelial cells through inhibition of the adhesins of Candida and inhibition of the hyphal transition. The results suggest that the effect of WG and of IY is in particular due to a mechanical effect through production of strong aggregation of Candida, which prevents adhesion of Candida to the epithelial cells. All these effects induce a considerable acceleration of the elimination of Candida, which leads to a low or even absent inflammatory response. It is interesting to note that, in all the determinations carried out, the fungal burden obtained after treatment with the yeast products tested (GI, IY, and WG) was comparable to that obtained with fluconazole, the standard antifungal used in the treatment of oropharyngeal candidiasis.

1-13. (canceled)

14. A method for preventing and/or treating oropharyngeal candidiasis in a subject, said method comprising a set of administering to said subject a Saccharomyces cerevisiae yeast strain deposited with the CNCM on Oct. 17, 2007 under Accession Number I-3856.

15. The method according to claim 14, wherein the Saccharomyces cerevisiae yeast strain is in the live form or in the inactive form.

16. The method according to claim 14, wherein the Saccharomyces cerevisiae yeast strain is in the form of dry yeast.

17. The method according to claim 16, wherein the Saccharomyces cerevisiae yeast strain in the form of dry yeast is in the form of active dry yeast.

18. The method according to claim 14, wherein the Saccharomyces cerevisiae yeast strain is in a fractionated form.

19. The method according to claim 18, wherein the fractionated form is selected from the group consisting of cell walls of said yeast, β-glucans from said cell walls of said yeast, wall mannoproteins of said yeast, extracts from said yeast, and combination thereof.

20. The method according to claim 14, wherein the effective amount of the Saccharomyces cerevisiae yeast strain number I-3856 is comprised in a pharmaceutical composition, which further comprises at least one physiologically acceptable excipient.

21. The method according to claim 20, wherein the pharmaceutical composition is intended for topical administration or for administration by the oral route.

22. The method according to claim 20, wherein the pharmaceutical composition further comprises at least one additional active pharmaceutical ingredient having a soothing, anti-irritant, analgesic, anti-inflammatory, wound-healing, antibiotic, antipyretic, or antifungal activity.

23. The method according to claim 22, wherein the pharmaceutical composition is intended for topical administration or for administration by the oral route.

24. The method according to claim 21, wherein the pharmaceutical composition further comprises at least one additional active pharmaceutical ingredient having a soothing, anti-irritant, analgesic, anti-inflammatory, wound-healing, antibiotic, antipyretic, or antifungal activity.

25. The method according to claim 20, wherein the pharmaceutical composition is in the form of a toothpaste, a mouthwash, an oral spray, a cream or an oral gel, an orodispersible stick, a stick to be diluted in water, or a vial with a push-button stopper.

26. The method according to claim 21, wherein the pharmaceutical composition is in the form of a toothpaste, a mouthwash, an oral spray, a cream or an oral gel, an orodispersible stick, a stick to be diluted in water, or a vial with a push-button stopper.

27. The method according to claim 22, wherein the pharmaceutical composition is in the form of a toothpaste, a mouthwash, an oral spray, a cream or an oral gel, an orodispersible stick, a stick to be diluted in water, or a vial with a push-button stopper.

27. The method according to claim 23, wherein the pharmaceutical composition is in the form of a toothpaste, a mouthwash, an oral spray, a cream or an oral gel, an orodispersible stick, a stick to be diluted in water, or a vial with a push-button stopper.

28. The method according to claim 14, wherein the oropharyngeal candidiasis is a side effect of a medical treatment, or the oropharyngeal candidiasis is present or likely to develop in a patient in an immunocompromised state.

29. The method according to claim 14, wherein the oropharyngeal candidiasis is present in an infant or an elderly person.

30. The method according to claim 14, wherein administering the Saccharomyces cerevisiae yeast strain further prevents or inhibits the spread of Candida infection to the esophagus, the stomach or the small intestine in the subject with oropharyngeal candidiasis.

31. A method for preventing or inhibiting the spread of Candida infection to the esophagus, the stomach or the small intestine in a patient with oropharyngeal candidiasis, the method comprising a step of administering to said patient a Saccharomyces cerevisiae yeast strain deposited with the CNCM on Oct. 17, 2007 under Accession Number I-3856.
